(19) United States
(12) Patent Application Publication
(10) Pub. No.: US 2007/0264637 A1
(43) Pub. Date: Nov. 15, 2007

(54) 11q DELETION AS A MOLECULAR GENETIC MARKER IN BREAST CANCER

(75) Inventors: Joan Climent-Bataller, Valencia (ES); Jose Ãngel Martinez-Climent, Pamplona (ES); Daniel Pinkel, Lafayette, CA (US); Donna G. Albertson, Lafayette, CA (US)

Correspondence Address: MOORE & VAN ALLEN PLLC, P.O. BOX 13706, Research Triangle Park, NC, 27709, US

(73) Assignees: PROYECTO DE BIOMEDICINA CIMA, S.L., Pamplona-Navarra (ES); THE REGENTS OF THE UNIVERSITY OF CALIFORNIA, Oakland, CA (US)

(21) Appl. No.: 11/382,999

(22) Filed: May 12, 2006

(51) Int. Cl. C12Q 1/68

(52) U.S. Cl. 435/6

(57) ABSTRACT

The present invention relates to methods for predicting sensitivity and response to a chemotherapy in a patient suffering from breast cancer based on the detection of the presence or absence of a deletion in the human chromosome region 11q21-q25 in a breast tumor sample from said patient.

### FIELD OF THE INVENTION

The present invention relates to methods for predicting sensitivity and response to a chemotherapy in a patient suffering from breast cancer based on the detection of the presence or absence of a deletion in the human chromosome region 11q21-q25 in a breast tumor sample from said patient.

### BACKGROUND OF THE INVENTION

Breast cancer is the leading cause of cancer deaths among women. There are over 1 million cases of breast cancer per year on a global basis, of which around 0.5 million are in the US.

Patients diagnosed with early breast cancer have greater than a 90% 5 year relative survival rate, as compared to 20% for patients diagnosed with distally metastasised breast cancer. Therefore, current screening programs aim to identify breast cancer in its earliest stages of development. Nowadays, localized breast cancer (confined to the mammary gland) represents around 70-75% of the cases diagnosed in the clinic. These local carcinomas are usually treated with surgery, followed in some cases by local radiotherapy, after which the administration of systemic adjuvant chemotherapy is considered as an option. This individualized decision is based on the consensus criteria of Saint Gallen, which evaluate age, tumor size, histological grade and subtype, hormone receptor status and HER2neu oncogene expression.

It is now well established that adjuvant systemic chemotherapy and/or endocrine therapy result in significant improvement of clinical outcome in patients with breast cancer disseminated to the lymph nodes. In lymph-node negative breast cancer (NNBC), systemic therapy is also recommended in a large fraction of patients. In women with hormone-receptor positive disease, endocrine therapy with tamoxifen or novel aromatase inhibitors should be considered for most if not all patients. However, the group of patients with NNBC that will obtain clinical benefit from the use of adjuvant chemotherapy is still a problematic debate. In patient with young age, with large sized or high pathological grade tumors, or with hormone receptor-negative tumors, systemic chemotherapy improves the odds of disease-free and overall survival. Among the different chemotherapy regimens, those containing anthracyclines (adriamycine or epirubicine) are on average more effective. Therapy with trastuzumab seems to be effective in the subset of HER2 positive NNBC. However, despite obvious therapeutic advances, approximately one fourth of NNBC patients will have tumor recurrence (metastasis) that is potentially treatable but ultimately fatal. In addition, and because surgery alone is curative in approximately 70% of NNBC patients, clinicians are faced with the dilemma of possible over-treatment of women who would have been cured without any systemic therapy. These data highlight the need for more sensitive and specific therapy-predictive indicators to refine the use of the multiple treatment options.

A number of individual biological markers have been used to improve patient stratification based on risk recurrence. However, breast cancer is a multi-factorial disease characterized by the accumulation of numerous molecular alterations in the cells, thus indicating that response to treatment is not likely associated with the mutation of a single gene but rather with the concurrent disturbance of many genes. Gene expression profiling enables the characterization of the variation in the transcriptional program in breast tumors by measuring expression of thousands of mRNAs in tissue specimens simultaneously. Using this technology, van't Veer et al. (van't Veer L J et al. Gene expression profiling predicts clinical outcome of breast cancer. Nature 2002;415(6871):530-6) reported a gene-expression signature of breast tumor cells that was a more powerful predictor of disease outcome than standard clinical and histological criteria. An increasing number of studies have subsequently profiled breast tumor specimens using distinct microarray platforms as well as RT-PCR techniques, reporting unique gene expression profiles correlated with poor outcome. However, the apparent variability and lack of reproducibility observed among these previous transcriptional analyses and the requirements for high-quality RNA obtained from fresh tissues have limited their application to the clinical setting.

Four reports have evaluated the influence of del(11q) in clinical outcome of breast cancer patients. In 1995, Winquist et al. (Winqvist R, et al. Loss of heterozygosity for chromosome 11 in primary human breast tumors is associated with poor survival after metastasis. Cancer Res. 1995;55:2660-2664) analyzed a series of breast carcinomas from 86 unselected patients for loss of heterozygosity (LOH) of chromosome 11q, reporting that the presence of LOH in 11q was associated with inferior survival. Gentile et al. (Gentile M, Olsen K, Dufmats M, Wingren S. Frequent allelic losses at 11q24.1-q25 in young women with breast cancer: association with poor survival. Br J Cancer. 1999;80:843-849) studied the presence of 11q deletion in 102 young patients (aged less than 37) with breast cancer, not confirming the association of this marker with worse clinical outcome. However, Laake et al. evaluated a large series of 918 unselected breast cancer biopsies in a multicentric study and reported that the LOH of chromosome 11q (around ATM gene) was an indicator of reduced survival (Laake K, et al. Loss of heterozygosity at 11q23.1 and survival in breast cancer: results of a large European study. Breast Cancer Somatic Genetics Consortium. Genes Chromosomes Cancer. 1999;25:212-221). More recently, Chunder and cols. (Chunder N, et al. Analysis of different deleted regions in chromosome 11 and their interrelations in early- and late-onset breast tumors: association with cyclin D1 amplification and survival. Diagn Mol Pathol. 2004;13:172-182) did not confirm such findings in a series of breast cancer patients. These reports included patients with local and advanced disease and what is more important, none of these studies had into account the therapy administered to the patients.

Nowadays, there is a well accepted need for new molecular genetic markers that accurately predict response to the different available treatment options in cancer. This is especially true for early breast cancer, primarily because of its frequency in the clinical practice.

### SUMMARY OF THE INVENTION

The present invention relates to a method for predicting sensitivity and response to a chemotherapy in a patient with a breast cancer, said method comprising determining the presence or absence of a deletion in the human chromosome region 11q21-q25 in a breast tumor sample from said patient.

Using novel comparative genomic hybridization to BAC microarrays (array CGH), the inventors of the present invention have identified a single genetic marker, the deletion of chromosome 11q-del(11q21-q25)—that predicts response to chemotherapy in early breast cancer. As it is shown in the Example accompanying the present application, early breast cancer patients with 11q deleted tumors can benefit from the use of systemic chemotherapy which could be considered then as the first treatment option despite other standard clinical, histopathological and genetic characteristics.

The present invention demonstrates that the deletion of chromosome 11q may represent one of the initial examples of the application of novel genetic markers to predict sensitivity to standard therapies in cancer. Particularly, the inventors have shown that those early breast cancer patients with 11q deleted tumors benefit from the use of anthracycline-based chemotherapy.

Thus, in one aspect, the invention relates to an in vitro method for predicting sensitivity and response to chemotherapy in a patient with a breast cancer, said method comprising:
providing a breast tumor sample from said patient;
obtaining a nucleic acid present in said sample; and
detecting the presence or absence of a deletion in the human chromosome region 11q21-q25;
   wherein the presence of said deletion is indicative of a favourable predisposition of said patient to respond to a chemotherapy treatment, and wherein the presence of said deletion may be used to design an individual chemotherapy treatment for said patient, and/or to minimize the relapse risk by administering chemotherapy treatment to said patient, and/or to increase the survival rate of said patient by administering chemotherapy treatment to said patient.

In a second aspect, the invention refers to a kit for predicting sensitivity and response to chemotherapy in a patient with a breast cancer, said kit comprising one or more nucleic acid probes each of which selectively binds to a target polynucleotide sequence on the chromosome region 11q21-q25, under conditions in which the probe forms a stable hybridization complex with the target polynucleotide sequence.

### BRIEF DESCRIPTION OF THE FIGURES

FIG. 1 is a representation and description of genome-wide array CGH technique: hybridization, imaging and analytical procedures.

FIG. 2A shows a boxplot representing differences between presentation age in both treatment groups (p=0.003). Kapplan Meier curves for (FIG. 2B) Disease Free Survival (DFS) and (FIG. 2C) Overall Survival (OS) in AC group and non-AC group.

FIG. 3 is a representation of array CGH results of 185 NNBC. Genomic gains and losses are depicted in light grey (right) and dark grey (left), respectively.

FIG. 4 shows a comparison of array CGH and CGH to metaphase chromosome techniques in 44 NNBC samples.

FIG. 5 shows the correlation of ER/PR expression status with genomic data. FIG. 5A shows the genomic gain of BAC clones mapped to 1q21-q43 and to 16p12 chromosome regions and the genomic loss of clones mapped to 16q21-q24 were associated with positive ER (marked with arrowheads). FIG. 5B shows that the progesterone receptor (PR) status is not significantly associated with abnormalities of 1q, 16p or 16q arms, but a borderline significance in chromosome 16 is observed. Instead, the genomic loss of clones mapped to 4p13-16 and 5q11.2-q31 were observed associated statistically with negative PR (marked with arrowheads). FIG. 5C shows the frequency plot of all BAC clones in chromosome 16 comparing ER positive (FIG. 5C2) versus ER negative (FIG. 5C1) tumors. FIG. 5D is a representation of log2ratios from clones in chromosome 16 in one ER-positive breast tumor.

FIG. 6 shows an association of genomic results with clinical outcome in NNBC patients. In FIG. 6A is presented that in the AC group, after adjustment for multiple testing, none of the 2,460 BAC clones was associated with tumor relapse. In FIG. 6B it is shown that in the non-AC group, however, there were statistically significant differences (p<0.05) in 8 BAC clones, clustered to the long arm of chromosome 11, that showed more common deletion in the group of tumor recurrences. Kaplan Meier curves showed differences in disease-free survival (DFS) for 11q deleted tumors vs. those without deletion in the non-AC group (DFS±ES at 10 years, 40±14% vs. 86±6%, p<0.0001) but not in the non-AC group.

FIG. 7A is a representation of the region of deletion in chromosome 11q. The eight clones clustered to chromosome 11 from bands 11q23.1 to 11q24.1 are highlighted in yellow. When the adjusted value for statistical significance was of <0.1 instead of <0.05, the number of BAC clones correlated with relapse increased to 24, all of them mapped to 11q21-q25. FIG. 7B is an array CGH (left) and CGH to chromosome analyses of a breast cancer biopsy showing concomitant cyclin D1 amplification at chromosome band 11q13 and genomic deletion of 11q21-q25.

FIG. 8 shows the correlation of 11q deletion in the test and validation series. In FIG. 8A the bars show the comparative relapse rates of tumors with 11q vs. non-deleted in 185 NNBC patients (training set). FIG. 8B shows the relapse rates in the validation set of 88 NNBC patients. In FIG. 8C it is shown the distribution of recurrences between the 11q vs. non-11q subgroups is shown in the training and validation sets. In FIG. 8D Kaplan-Meier curves show differences in DFS for the validation group, resembling the data obtained in the training set of 185 patients.

### DETAILED DESCRIPTION OF THE INVENTION

Using a novel comparative genomic hybridization assay (array CGH), the inventors of the present invention have now identified a single genetic marker, the deletion of chromosome 11q-del(11q21-q25)—that predicts response to chemotherapy in breast cancer. Particularly, the inventors have shown (see Example 1) that those early breast cancer patients with 11q deleted tumors benefit from the use of anthracycline-based chemotherapy. These data disclose that the deletion of chromosome 11q represent one of the first examples of the application of novel genetic markers to predict sensitivity to standard therapies in cancer.

Therefore, in one aspect, the invention relates to an in vitro method for predicting sensitivity and response to chemotherapy in a patient with a breast cancer, said method comprising:
providing a breast tumor sample from said patient;
obtaining a nucleic acid present in said sample; and
detecting the presence or absence of a deletion in the human chromosome region 11q21-q25;
   wherein the presence of said deletion is indicative of a favourable predisposition of said patient to respond to a chemotherapy treatment, and wherein the presence of said deletion may be used to design an individual chemotherapy treatment for said patient, and/or to minimize the relapse risk by administering chemotherapy treatment to said patient, and/or to increase the survival rate of said patient by administering chemotherapy treatment to said patient.

In a particular embodiment of the invention, the deletion to be determined is located at the human chromosome region 11q23.1-q24.1.

Tissue Samples

In order to carry out the method of the invention, a sample is obtained from the subject under study. In a particular embodiment, said sample is a tumour tissue sample or portion thereof. In a more particular embodiment, said tumor tissue sample is a breast tumor tissue sample from a patient suffering from breast cancer. Said sample can be obtained by conventional methods, e.g., biopsy, by using methods well known to those of ordinary skill in the related medical arts. Methods for obtaining the sample from the biopsy include gross apportioning of a mass, or microdissection or other art-known cell-separation methods. Tumour cells can additionally be obtained from fine needle aspiration cytology.

Samples can be obtained from subjects previously diagnosed or not with breast cancer, or from subjects who are receiving or have previously received anti-breast cancer treatment. In a particular embodiment, samples can be obtained from patients who have not previously received any anti-breast cancer treatment.

In order to simplify conservation and handling of the samples, these can be formalin-fixed and paraffin-embedded or first frozen and then embedded in a cryosolidifiable medium, such as OCT-Compound, through immersion in a highly cryogenic medium that allows for rapid freeze.

In a particular embodiment, the presence of the mutation is determined using nucleic acids obtained from as fresh tissue from a biopsy or fine needle aspiration cytology. Other tissue samples are envisaged, such a formalin-fixed, paraffin-embedded tissue sample depending on their availability.

Fixed and paraffin-embedded tissue samples are broadly used storable or archival tissue samples in the field of oncology. Nucleic acid may be isolated from an archival pathological sample or biopsy sample which is first deparaffinized. An exemplary deparaffinization method involves washing the paraffinized sample with an organic solvent, such as xylene, for example. Deparaffinized samples can be rehydrated with an aqueous solution of a lower alcohol. Suitable lower alcohols, for example include, methanol, ethanol, propanols, and butanols. Deparaffinized samples may be rehydrated with successive washes with lower alcoholic solutions of decreasing concentration, for example. Alternatively, the sample is simultaneously deparaffinized and rehydrated. The sample is then lysed and nucleic acid is extracted from the sample. As an illustrative, non limitative example, tissue selected for fixation and paraffin embedding can be fixed in 10% buffered formalin for 16 hours to 48 hours. After this period of time, said tissue will be embedded in paraffin following conventional techniques. Nevertheless, nucleic acid quality issues are especially delicate when analyzing formalin-fixed tissue samples.

In a particular embodiment, the presence of the mutation is determined using nucleic acids obtained from a biopsy tissue sample or fine needle aspiration cytology. Because of the variability of the cell types in diseased-tissue biopsy material, and the variability in sensitivity of the diagnostic methods used, the sample size required for analysis may range from 1, 10, 50, 100, 200, 300, 500, 1,000, 5,000, 10,000, to 50,000 or more cells. The appropriate sample size may be determined based on the cellular composition and condition of the biopsy or cytology, and the standard preparative steps for this determination and subsequent isolation of the nucleic acid for use in the invention are well known to one of ordinary skill in the art.

DNA Extraction and Amplification

Using standard methods, the biological sample may be treated to physically or mechanically disrupt tissue or cell structure, to release intracellular components into an aqueous or organic solution to prepare nucleic acids for further analysis. The nucleic acids are extracted from the sample by procedures known to the skilled person and commercially available. In a particular embodiment, the total DNA extracted from tissue samples will represent the working material suitable for subsequent detection of the genetic marker of interest.

The term “nucleic acid” refers to a multimeric compound comprising nucleosides or nucleoside analogues which have nitrogenous heterocyclic bases, or base analogues, which are linked by phosphodiester bonds to form a polynucleotide.

The term “DNA” refers to deoxyribonucleic acid. A DNA sequence is a deoxyribonucleic sequence. DNA is a long polymer of nucleotides and encodes the sequence of the amino acid residues in proteins using the genetic code.

Once the sample has been obtained and the total DNA has been extracted, amplification of nucleic acid may be carried out in order to produce sufficient sample material for further detection procedures. Several techniques can be used for producing sufficient starting material. These techniques include polymerase chain reaction (PCR), degenerate primer PCR using one or several sets of primers, rolling circle amplification, etc. Examples of techniques sufficient to direct persons of skill through in vitro amplification methods are found in Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

In a particular embodiment, the amplification of the DNA is carried out by means of PCR. The general principles and conditions for amplification and detection of nucleic acids, such as using PCR, are well known for the skilled person in the art.

Detection of DNA Mutation

Detection of DNA sequence mutations may proceed by any of a number of methods known to those skilled in the art (Kilger et al., 1997, Nucleic Acids Res. 25: 2032-4). In general, DNA sequence mutations may be detected directly by nucleic acid sequencing methods such as cycle sequencing or direct dideoxynucleotide sequencing, in which some or the entire DNA of interest that has been harvested from the tissue sample is used as a template for sequencing reactions. An oligonucleotide primer or set of primers specific to the gene or DNA of interest is used in standard sequencing reactions. Other methods of DNA sequencing, such as sequencing by hybridization, sequencing using a “chip” containing many oligonucleotides for hybridization, sequencing by HPLC, and modifications of DNA sequencing strategies such as multiplex allele-specific diagnostic assay, dideoxy fingerprinting, and fluorogenic probe-based PCR methods and cleavase-based methods may be used.

Alternatively, detection can be carried out using primers that are appropriately labelled, and the labeled products can be detected using procedures and equipment for detection of the label.

In a particular embodiment, the determination of the chromosome 11q deletion status can be measured in the DNA obtained from the tumor cells according to standard procedures such as quantitative PCR or comparative genomic hybridization to microarray technologies; or in the tumor cells from the paraffined-embedded section or from the cytology preparation by FISH using appropriate molecular probes. In a particular embodiment, the detection of a deletion in the human chromosome region 11q21-q25 is carried out by a hybridization-based assay. In a particular embodiment, the detecting step of the method of the invention comprises contacting the nucleic acid sample with one or more nucleic acid probes each of which selectively binds to a target polynucleotide sequence on the chromosome region 11q21-q25, under conditions in which the probe forms a stable hybridization complex with the target polynucleotide sequence; and detecting the hybridization complex. In a particular embodiment, the nucleic acid probes used in the method of the present invention are labelled with a fluorophore.

In a particular embodiment, the step of detecting the hybridization complex comprises determining the copy number of the target polynucleotide sequence, thereby determining the presence of the deletion.

In a preferred embodiment of the invention, said hybridization-based assay is selected from the group consisting of Southern blot, LOH (loss of heterozygosity), PCR, in situ hybridization (ISH) fluorescence ISH (FISH) and comparative genomic hybridization (CGH). In a more preferred embodiment, the method is a comparative genomic hybridization assay.

In a particular embodiment of the invention, said hybridization-based assay is an array-based assay. In a preferred embodiment, said hybridization assay is an array-based CGH assay.

In a particular embodiment, once the sample has been obtained and the total DNA has been extracted, genome-wide analysis of DNA copy number changes by comparative genomic hybridization (CGH) is carried out. In general, for a typical CGH measurement, total genomic DNA is isolated from test and reference cell populations, differentially labeled and hybridized to a representation of the genome that allows the binding of sequences at different genomic locations to be distinguished. Hybridization reactions can be performed under conditions of different stringency. The stringency of a hybridization reaction includes the difficulty with which any two nucleic acid molecules will hybridize to one another. For any hybridization, stringency can be varied by manipulation of three factors: temperature, salt concentration, and formamide concentration. High temperature and low salt increases stringency. Formamide decreases melting point of DNA, thus lowering the temperature at which a hybrid between two nucleic acid molecules forms. Preferably, each hybridizing polynucleotide hybridizes to its corresponding polynucleotide under reduced stringency conditions, more preferably stringent conditions, and most preferably highly stringent conditions.

The amount of specimen DNA is frequently a constraint on CGH measurements. Typical array CGH procedures use 300 ng to 3 μg of specimen DNA in the labelling reaction, equivalent to approximately 50.000 to 500.000 mammalian cells. Usually, random primer labeling protocols are employed, which also amplifies the DNA, so that several micrograms are used in the hybridization.

Array CGH has been implemented using a wide variety of techniques. In a particular embodiment, array CGH is carried out using arrays from large-insert genomic clones such as bacterial artificial chromosomes (BACs). The general principles and conditions for detection of nucleic acids, such as using array CGH (comparative genomic hybridization (CGH) to BAC microarrays), are well known for the skilled person in the art. This technique allows scanning the entire genome for DNA copy number changes therefore allowing quantitative detection of DNA copy number variation in tumor genomes with high resolution (Pinkel D, et al. High resolution analysis of DNA copy number variation using comparative genomic hybridization to microarrays. Nat Genet 1998;20(2):207-11 and Hodgson G, et al. Genome scanning with array CGH delineates regional alterations in mouse islet carcinomas. Nat Genet 2001;29(4):459-64).

As an illustrative non limitative example, in the array CGH carried out by the method of the present invention test tumor and reference genomic DNAs can be labeled by random priming using Cy3 and Cy5 fluorophores. Then, the images of the arrays may be analysed using, for example, a cooled coupled device (CCD) camera and appropriate software. The general conditions for the array CGH of the method of the present invention are as illustrated in the Example 1 of the description.

The major technical challenge of array CGH is generating hybridization signals that are sufficiently intense and specific so that copy number changes can be detected. The signal intensity on an array element is affected by a number of factors including the base composition, the proportion of repetitive sequence content, and the amount of DNA in the array element available for hybridization.

Array elements made from genomic BAC clones typically provide more intense signals than elements employing shorter sequences such as cDNAs, PCR products, and oligonucleotides. The higher signals form the more complex array elements result in better measurement precision, allowing detection of single-copy transition boundaries-even in specimens with a high proportion of normal cells.

Anthracycline-Based Chemotherapy

As mentioned above, the inventors have shown that patients with early breast cancer with 11q deleted tumors benefit from the use of anthracycline-based chemotherapy. Therefore, in a particular embodiment, the method of the present invention specially refers to a method for predicting sensitivity and response to chemotherapy in a patient with a breast cancer, said method comprising the steps as described above, wherein the presence of said deletion is indicative of a favourable predisposition of said patient to respond to an anthracycline-based chemotherapy treatment, and wherein the presence of said deletion may be used to design an individual anthracycline-based chemotherapy for said patient, and/or to minimize the relapse risk by administering an anthracycline-based chemotherapy to said patient, and/or to increase the survival rate of said patient by administering an anthracycline-based chemotherapy to said patient.

Anthracycline antibiotics are an important group of antitumor drugs widely used in cancer chemotherapy. They are made from natural products produced by species of the soil fungus Streptomyces, and derivatives/analogues of these natural products. These drugs act during multiple phases of the cell cycle and are considered cell-cycle specific. Their principal mode of action is based on their interaction with DNA by binding and inserting between DNA bases, leading to chromatin unfolding and aggregation. These chromatin structural changes primarily interfere with DNA replication and transcription, thus leading to the apoptosis undergone by the cells treated with anthracyclines. The most frequently used anthracyclines are: doxorubicin, daunorubicin, epirubicin, mitoxantrone, and idarubicin.

As used herein, the term “anthracycline-based chemotherapy” refers to any type of chemotherapy including any of the anthracyclines, at any dose and dosage form.

Delivery Routes and Dosages

Chemotherapy compounds may be formulated for oral delivery or parenteral delivery. Furthermore, the chemotherapy compounds are formulated for delivery by a route selected from the group consisting of intravenous, intramuscular, oral, subcutaneous, intrathecal, intracranial and intraventricular.

Said drugs can be administered at different dosages. As an illustrative, non limitative, example, the anthracycline doxorubicin can be administered at a dose of 50 to 75 mg/m² of body surface area over about a 30-minute period, with about daily to four weekly doses, with courses repeated about every 21 to 30 days for four cycles. The dose of administration chosen can be given for up to about 7 weeks, according to this treatment regimen, or until undesirable side effects are observed.

The administration step is typically repeated on a cyclic basis, which may be repeated as appropriate over for instance 1 to 20 cycles. The cycle includes a phase of administering chemotherapy compounds, and usually also a phase of not adminsitering chemotherapy compounds. Typically the cycle is worked out in weeks, and thus the cycle normally comprises one or more weeks of a chemotherapy treatment phase, and one or more weeks to complete the cycle. A cycle of 3 weeks is preferred, but alternatively it can be from 2 to 6 weeks. The administration phase can itself be a single administration in each cycle of 1 to 72 hours, more usually of about 1, 3 or 24 hours; or an administration on a daily basis in the administration phase of the cycle for preferably 1 to 5 hours, especially 1 or 3 hours; or an administration on a weekly basis in the administration phase of the cycle for preferably 1 to 3 hours, especially 2 or 3 hours. A single administration at the start of each cycle is preferred. Preferably the administration time is about 1, 3 or 24 hour.

It will be recognized by one of skill in the art that the optimal quantity and spacing of individual dosages of a chemotherapy compound of the invention will be determined by extent of the disorder being treated, the form, route and site of administration, and the particular patient being treated. It will also be appreciated by one of skill in the art that the optimal course of treatment, i.e., the number of doses of an a chemotherapy compound of the invention given per administration treatment for a defined number of days or weeks, may be ascertained by those skilled in the art using conventional course of treatment determination tests.

In general, compositions for parenteral delivery will commonly comprise a solution of any chemotherapy compound, including the anthracyclines described above dissolved in an acceptable carrier, preferably an aqueous carrier. A variety of aqueous carriers may be employed, e.g., water, buffered water, 0.4% saline, 0.3% glycine, and the like. These solutions are sterile and generally free of particulate matter. These solutions may be sterilized by conventional, well-known sterilization techniques. The compositions may contain pharmaceutically acceptable auxiliary substances as required to approximate physiological conditions such as pH adjusting and buffering agents, etc. The concentration of the chemotherapy compound in such compositions may vary widely, i.e., from less than about 0.5%, usually at or at least about 1% to as much as 15 or 20% by weight, and will be selected primarily based on fluid volumes, viscosities, etc., according to the particular mode of administration selected.

Thus, as an illustrative, non limitative, example, the composition for intramuscular injection could be prepared to contain 1 ml sterile buffered water, and a therapeutically effective amount of the chemotherapy compound. Similarly, a composition for intravenous infusion could be made up to contain 250 ml of sterile Ringer's solution, and a therapeutically effective amount of the chemotherapy compound. Actual methods for preparing parenterally administrable compositions are well-known or will be apparent to those skilled in the art, and are described in more detail in, e.g., Remington's Pharmaceutical Science, 15th ed., Mack Publishing Company, Easton, Pa. The therapeutically effective amount of the chemotherapy compound can be determined by one skilled in the art. The age and weight of the patient, the stage of the disease, the mode of administration, the actual formulation and the possible coadministration of other therapeutic agents must all be taken into account when determining the optimal therapeutically effective amount of the chemotherapy compound to be administered.

The chemotherapy compound, such as anthracycline, may be lyophilized for storage and reconstituted in a suitable carrier prior to use.

Combination Therapy

In addition, any chemotherapy compound, such as anthracyclines, can be administered alone or frequently in combination with other chemotherapy compounds or drugs. The identity of the other drugs is not particularly limited, and suitable candidates include: a) drugs with antimitotic effects, especially those which targetcytoskeletal elements, including microtubule modulators such as taxane drugs (such as taxol, paclitaxel, taxotere, docetaxel), podophylotoxins or vinca alkaloids (vincristine, vinblastine); b) antimetabolite drugs (such as 5-fluorouracil, cytarabine, gemcitabine, purine analogues such as pentostatin, methotrexate); c) alkylating agents or nitrogen mustards (such as nitrosoureas, cyclophosphamide or ifosphamide); d) drugs which target topoisomerases such as etoposide; hormones and hormone agonists or antagonists such as estrogens, antiestrogens (tamoxifen and related compounds) and androgens, flutamide, leuprorelin, goserelin, cyprotrone or octreotide; e) drugs which target signal transduction in tumour cells including antibody derivatives such as herceptin; f) alkylating drugs such as platinum drugs (cis-platin, carbonplatin, oxaliplatin, paraplatin) or nitrosoureas; g) drugs potentially affecting metastasis of tumours such as matrix metalloproteinase inhibitors; h) gene therapy and antisense agents; i) antibody therapeutics; j) steroid analogues, in particular dexamethasone; k) anti-inflammatory drugs, including nonsteroidal agents (such as acetaminophen or ibuprofen) or steroids and their derivatives in particular dexamethasone; and l) anti-emetic drugs, including 5HT-3 inhibitors (such as palonisetron, gramisetron or ondasetron).

The National Institutes of Health (NIH) consensus conference convened in November 2000, and recommended adjuvant polychemotherapy for women with primary breast cancer greater than 1 cm in size, regardless of nodal, menopausal or hormonal receptor status. The NIH consensus also recommended four to six courses of chemotherapy. The 2005 St Gallen consensus recommends chemotherapy for endocrine nonresponsive disease and, primarily, endocrine therapy for endocrine-responsive disease. It also recommends considering the addition of chemotherapy for some intermediate and all high-risk patients in this group.

There are several adjuvant chemotherapy regimens that are used commonly in the USA and Europe. These are summarized in the recent review by Dr. Dang (Expert Review of Anticancer Therapy; March 2006, Vol. 6, No. 3, Pages 427-436). The most commonly used include four cycles of doxorubicin/cyclophosphamide (AC), four cycles of AC followed by four cycles of paclitaxel, docetaxel (T)+AC (TAC), FEC or CEF (5-fluorouracil, epirubicin, cyclophosphamide) for six cycles, and FAC or CAF (5-fluorouracil/doxorubicin/cyclophosphamide) for six cycles. Cyclophosphamide, methotrexate and 5-fluorouracil (CMF) has generally been reserved for low-risk, node-negative breast cancer treatment. The taxanes, paclitaxel and docetaxel, are well established in metastatic breast cancer treatment and can increase response rate and duration of response. Taxanes lack cross-resistance with anthracyclines and are therefore quickly deemed worthwhile for evaluation in the adjuvant setting. Results from several randomized trials demonstrate a benefit from the addition of a taxane to an anthracycline-based regimen.

When coadministered with one or more chemotherapy compounds, the chemotherapy compound may be administered either simultaneously with the second agent, or separately, e.g., sequentially. If administered separately, e.g., sequentially, the attending physician will decide on the appropriate sequence of administering the chemotherapy compound in combination with other agents.

Depending on the developmental stage of the disease, said treatments would be useful in preventing the risk of developing tumors, in promoting tumor regression, in stopping tumor growth and/or in preventing metastasis.

Although guidance for the dosage is given above, the correct dosage of the chemotherapy compound will vary according to the particular formulation, the mode of application, and the particular situs, host and tumor being treated. Other factors like age, body weight, sex, diet, time of administration, rate of excretion, condition of the host, drug combinations, reaction sensitivities and severity of the disease shall be taken into account.

The use of anthracyclines is particularly preferred for the treatment of breast cancer. Thus, in a particular embodiment, the method of the invention further comprises considering the data obtained for designing an individual chemotherapy treatment for said patient based on an anthracycline-based chemotherapy.

It is widely held that the most common breast cancer initiates as the pre-malignant stage of atypical ductal hyperplasia (ADH), progresses into the pre-invasive stage of ductal carcinoma in situ (DCIS), and culminates in the potentially lethal stage of invasive ductal carcinoma (IDC). It starts in a milk passage, or duct, of the breast, which has broken through the wall of the duct, and invaded the fatty tissue of the breast. At this point, it can metastasize, or spread to other parts of the body through the lymphatic system and bloodstream. About 80% of invasive breast cancers are infiltrating ductal carcinomas. Thus, in one particular embodiment, the patient is a patient with lymph-node negative breast cancer. In another particular embodiment, the patient is a patient with lymph-node positive breast cancer. In another particular embodiment, the patient is a patient with metastatic breast cancer.

If desired, the cancer screening methods of the present invention may be readily combined with other methods in order to provide an even more reliable indication of diagnosis or prognosis, thus providing a multi-marker test.

Kits

In another aspect, the invention refers to a kit for predicting sensitivity and response to chemotherapy in a patient with a breast cancer, said kit comprising one or more nucleic acid probes each of which selectively binds to a target polynucleotide sequence on the chromosome region 11q21-q25, under conditions in which the probe forms a stable hybridization complex with the target polynucleotide sequence. Said probe can be directly or indirectly labeled. Thus, in a particular embodiment of the invention, the probe is directly labeled. In another particular embodiment of the invention, the probe is indirectly labeled.

In a particular embodiment of the invention the nucleic acid probe is attached to a solid surface. In another particular embodiment, the attached probe is a member of a nucleic acid array.

In one particular embodiment of the invention, the kit further comprises informational material. The informational material can be descriptive, instructional, marketing, or other material that relates to the methods described herein and/or the use of said probes, for the methods described herein.

The informational material of the kits is not limited in its form. In one embodiment, the informational material can include information about production of the compounds, concentration, date of expiration, batch, or production site information, and so forth. In one embodiment, the informational material relates to teach that the detection of a deletion in the chromosome region 11q21-25 in a cell from a breast tumor sample of a patient is indicative of a favourable predisposition of said patient to respond to a chemotherapy treatment.

The kit can include one or more containers for the probe or probes. In some embodiments, the kit contains separate containers, dividers or compartments for the probes and the informational material. For example, the probe or probes can be contained in vials and the informational material can be contained in a plastic sleeve or packet. In other embodiments, the separate elements of the kit are contained within a single, undivided container. For example, the probes are contained in different vials that have attached thereto the informational material in the form of a label. In some embodiments, the kit includes a plurality (e.g., a pack) of individual containers. For example, the kit includes a plurality of vials for the different probes and informational material thereof.

The invention is further illustrated with the following Example, which is provided to illustrate certain embodiments of the present invention and is not to be construed as limiting the invention.

### EXAMPLE 1

### 11q Deletion as a Therapy-Predictive Indicator of Clinical Outcome in Early Breast Cancer

1. Materials and Methods

1.1 Study Design and Selection of Patients

Tumor biopsy specimens were obtained retrospectively from patients newly diagnosed of NNBC treated in the Department of Hematology and Medical Oncology, Hospital Clinico, University of Valencia (Spain), between September 1979 and June 2000. Patients were selected on the basis of the availability of frozen tumor biopsy samples and the following selection criteria:

1) diagnosis of primary invasive breast carcinoma of any size;

2) treatment by modified radical mastectomy or breastconserving surgery, including dissection of axillary lymph nodes, followed by radiotherapy if indicated;

3) the apical axillary lymph nodes were tumor-negative (pathological examination, pN0);

4) complete clinical data were available, and

5) infiltration of at least 50% of tumor cells in frozen tumor sections assessed by hematoxylin/eosin (H&E) staining.

A cohort of 185 patients fulfilled these criteria and was included into the study. The median age of the patients was 58 years (range, 21 to 86). Clinicopathological variables including tumor size, histological grade and subtype and estrogen receptor (ER) and progesterone receptor (PR) status were determined following standard methods as reported by Climent J, et al. (2002) Genomic loss of 18p predicts an adverse clinical outcome in patients with high-risk breast cancer. Clin Cancer Res; 8(12):3863-9. Ninety patients received adjuvant systemic chemotherapy consisting of anthracyclines, mainly doxorubicin (adriamycine), and cyclophosphamide, whereas 95 patients were not treated with adjuvant chemotherapy but with either hormonal therapy with tamoxifen (n=56) or did not receive additional therapy (n=39). Chemotherapy schemes are shown in Table 1. Informed consent was obtained from each subject or subject's guardian. Human investigations were performed after approval by an institutional review board on scientific and ethical affairs.

TABLE 1
Chemotherapeutic schemes of 185 breast cancer patients
AC PROTOCOL:

| | | |
|---|---|---|
| A.- | AD: 30 mg/m² IV day 1 | Every 21 days × |
| | CPM: 600 mg/m² IV day 1 | 3 cycles |
| | | (n = 17/90) |
| B.- | AD: 30 mg/m² IV day 1 | Every 21 days × |
| | CPM: 600 mg/m² IV day 1 | 8 cycles |
| | | (n = 43/90) |
| C.- | AD: 60 mg/m² IV day 1 | Every 21 days × |
| | CPM: 600 mg/m² IV day 1 | 4 cycles |
| | | (n = 20/90) |

FAC PROTOCOL

| | | |
|---|---|---|
| D.- | AD: 60 mg/m² IV day 1 | Every 21 days × |
| | CPM: 600 mg/m² IV day 1 | 4 cycles |
| | 5-FU: 600 mg/m² IV day 1 | (n = 6/90) |

CMF PROTOCOL

| | | |
|---|---|---|
| E.- | CPM: 600 mg/m² IV day 1 | Every 21 days × |
| | MTX: 40 mg/m² IV day 1 | 8 cycles |
| | 5-FU: 600 mg/m² IV day 1 | (n = 4/90) |

ADJUVANT CHEMOTHERAPY REGIMENES
AD: Adriamycin (Doxorrubicin)
CPM: Cyclophosphamide
5-FU: 5-Fluoracil (Fluorouracil)
MTX: Methothrexate

1.2 Microarray-Based Comparative Genomic Hybridization (Array CGH)
1.2.1 DNA Extraction Hybridization and Imaging

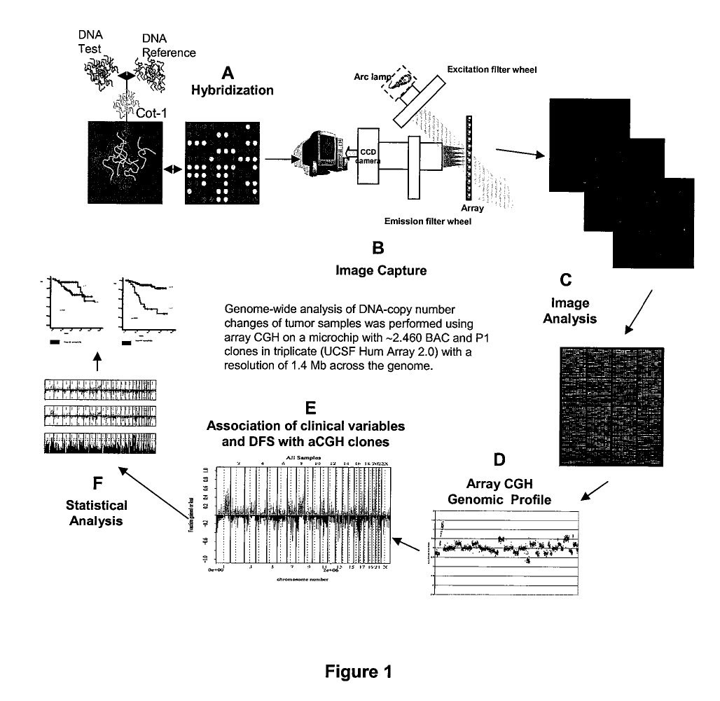

Previous to DNA extraction, hematoxylin/eosin (H&E) stained tumor sections were examined to select samples with more than 50% of tumoral cells. Around 20-30 sections of 25 μm were used for DNA extraction. After removing the OCT with PBS (Phosphate Buffered Saline) washes, DNA was extracted as previously described (Climent J, et al. (2002) Genomic loss of 18p predicts an adverse clinical outcome in patients with high-risk breast cancer. Clin Cancer Res;8(12):3863-9). Genome-wide analysis of DNA-copy number changes was performed using array CGH (comparative genomic hybridization (CGH) to BAC microarrays) on a microchip with 2.460 BAC (bacterial artificial clones) and PAC (PI-derived artificial chromosome (PAC) clones printed in triplicate (UCSF Hum Array 2.0) with a resolution of 1.4 Mb across the genome (Snijders A M, et al. Assembly of microarrays for genome-wide measurement of DNA copy number. Nat Genet 2001;29(3):263-4). Methods and analytical procedures have been described in detail by Rubio-Moscardo F, et al. Mantle-cell lymphoma genotypes identified with CGH to BAC microarrays define a leukemic subgroup of disease and predict patient outcome. Blood 2005;105(11):4445-54 and Snijders A M, et al. Assembly of microarrays for genomewide measurement of DNA copy number. Nat Genet 2001;29(3):263-4. Briefly, 0.5 μg of test (tumor) and referente genomic DNAs were labeled by random priming using Cy3 and Cy5, respectively. After 48 hour of hybridization, slides were washed and mounted with DAPI. The images of the arrays were captured using a CCD camera, and the “UCSF SPOT” 2.0 software (available at http://www.jainiab.org/downioads.htmi) was used to analyze the images and measure tumoral vs. control fluorescence intensity ratios that were converted to the log2 scale. A second program, the “UCSF SPROC” was used to associate clones with each spot and to create a mapping information file that allows the data to be plotted relative to the position of the BACs on the draft human genome sequence (http://qenome.cse.ucsc.edu; May 2004 freeze). A formal data filtering procedure was then performed, and a SPROC output file consisting of averaged ratios of the triplicate spots for each clone, standard deviations of the replicates and plotting positions for each clone on the array was obtained (FIG. 1). For visualization of genomic data, the TreeView program 1.60 (Stanford, Calif.) was used. To confirm array CGH data, CGH to chromosomes was performed in 44 biopsies included in the study.

1.2.2 Interphase FISH Analysis.

To confirm specific gains and losses of BAC clones observed in the array CGH analyses, fluorescence in situ hybridization (FISH) studies using individual BAC clones as probes on isolated nuclei from frozen tumor sections was performed using a reported technique (Siebert R, et al. Detection of deletions in the short arm of chromosome 3 in uncultured renal cell carcinomas by interphase cytogenetics. J Urol 1998; 160(2):534-9). The gene loci examined corresponded to 5 overrepresented and 4 deleted BAC clones, using appropriate centromeric probes as controls. A total of 100 cells were examined on each of the 22 tumors examined. These clones were obtained from RZPD German Resource Center (Berlin, Germany) or purchased from Vysis (Downers Grove, Ill., US).

1.3 Statistical Analysis

1.3.1 Array Preprocessing

In order to process the genomic data obtained with array CGH and to compare the genomic alterations with different clinical phenotypes, a previously described analytical model was used (Slamon D J, et al. Human breast cancer: correlation of relapse and survival with amplification of the HER-2/neu oncogene. Science 1987;235(4785):177-82). Clones with ratios missing in 2 or more replicate spots (out of 3) were excluded from further analysis, as well as when the standard deviation of the replicates log2 ratios was above 0.2. In addition, clones that were successfully mapped to May 2004 release of human genome sequence and were declared present in more than 75% of the samples were included in the final analysis. Duplicate clones were averaged. The final dataset contained 2117 unique BACs, and clone values were missing in a median of 5.4% of the samples. Arrays were normalized by subtracting the median of each array from the average log2 ratio for every clone.

1.3.2 Copy Number Changes Identification

The array CGH data were analyzed using Hidden Markov Model (HMM) as implemented in the Bioconductor package aCGH using the default tunning parameters (Slamon D J, et al. cited supra). Log2 ratios as ordered in the genome were segmented into regions of constant copy number. In addition, the HMM model was employed to impute missing values by using the estimated copy number ratio for the segment containing the clone(s) with missing values. Clones with missing values located between segmented regions were assigned the mean value of the segment that is closer in genomic distance. Thus, each clone was assigned a segment value referred to as its “smoothed” value. Median absolute deviation (MAD) of the difference between the observed and smoothed values was used to estimate the tumor-specific experimental variation. All of the tumors had MAD less than 0.22. Clones for each array were assigned into three groups: gained—the smoothed log2 ratio of a clone in a particular tumor was higher than 3 times the MAD; lost—when the smoothed log2 ratio was less than 3 times the—MAD; and finally, not changed—when the log2 ratio can not be assigned to the lost or gained groups.

1.3.3 Association of Copy Number with Phenotypes

Smoothed, imputed data was used to study association with the following phenotypes: age, tumor size, histological grade and subtype, stage, estrogen and progesterone receptor status, and recurrence/survival. For example, for the right censored data Cox proportional hazards model was used, where difference in survival given different baseline log2 ratio for a given clone was tested. By controlling the False Discovery Rate it was corrected for multiple hypotheses testing. Significance was claimed at the FDR<0.05, which corresponds to the expectation of at most 5% of false discoveries among the loci declared significant. In addition, difference in recurrence/survival outcome for patients subgroups defined by the treatment assigned to them was tested.

1.3.4 Cross-Tabulation of Clinical Variables

Fisher's two-sided exact test 2×2 crosstabs was used to compare genomic events or clinical variables among both groups of treatment. To evaluate differences in disease-free survival, Kaplan-Maier survival curves for the sets of patients were examined.

1.3.5 GO Validation

Finally, a statistical analysis to check if there were any gene Ontology (GO) categories that were enriched in the genes located in the region of deletion in chromosome 11q23-q24 with respect to the whole genome was performed.

1.4 Clinical Series for Validation of Array CGH Results

To validate the possible association of chromosome 11q deletion with increased relapse rate, this was tested in a validation group of 88 tumor biopsy samples from an independent cohort of NNBC patients. These were 18 Spanish patients treated in different Institutions within the Valencia area whose genomes were analyzed with array CGH as described above. In addition, data from 70 patients were obtained from a recently published series of American breast cancer patients analyzed using similar whole-genome array CGH techniques. All patients fulfilled the reported inclusion criteria of the study. Kaplan-Maier survival curves for the two sets of patients were evaluated. Clinicopathological characteristics of the validation series are shown in Table 2.

TABLE 2
Clinico-pathological characteristics of the 88 NNBC
patients and tumors in the validation series

| | | Validation Group | | Validation Group | |
|---|---|---|---|---|---|
| Clinical | | N° | % | N° | % |
| Follow-Up | | N = 18 | | N = 70 | |
| Months | Range | 18-232 | | 8-127 | |
| | Mean | 108 | | 69 | |
| Age (years) | range | 39-79 | | 34-82 | |
| | mean | 55 | 25% | 45 | 25% |
| | | 61 | 50% | 54 | 50% |
| | | 72 | 75% | 65 | 75% |
| | <35 | 0 | 0% | 1 | 1% |
| | 35-50 | 4 | 22% | 28 | 39% |
| | 51-55 | 0 | 0% | 10 | 14% |
| | >55 | 14 | 78% | 31 | 46% |
| Hormonal Status | | | | | |
| | Post- | 14 | 78% | No data | |
| | Pre- | 4 | 22% | | |
| | Peri | 0 | 0% | | |
| Tumor Size | | | | | |
| T1 | <2 cm | 5 | 28% | 41 | 58% |
| T2 | 2-5 cm | 11 | 61% | 27 | 39% |
| T3 | >5 cm | 2 | 11% | 2 | 3% |
| Histologic Type | | | | | |
| | CDI Intraductal | 2 | 10% | No data | |
| | Ductal Infilt | 14 | 78% | | |
| | Lobular Infilt | 1 | 6% | | |
| | Others | 1 | 6% | | |
| Hormonal Receptors | | | | | |
| | ER− | 4 | 22% | 24 | 33% |
| | ER | 14 | 78% | 46 | 67% |
| | PgR | 9 | 50% | 29 | 40% |
| | PgR | 9 | 50% | 41 | 60% |
| Treatment | AC | 2 | 11% | 25 | 35% |
| | Non AC | 16 | 89% | 45 | 65% |

2. Results
2.1 Characteristics of the Patients

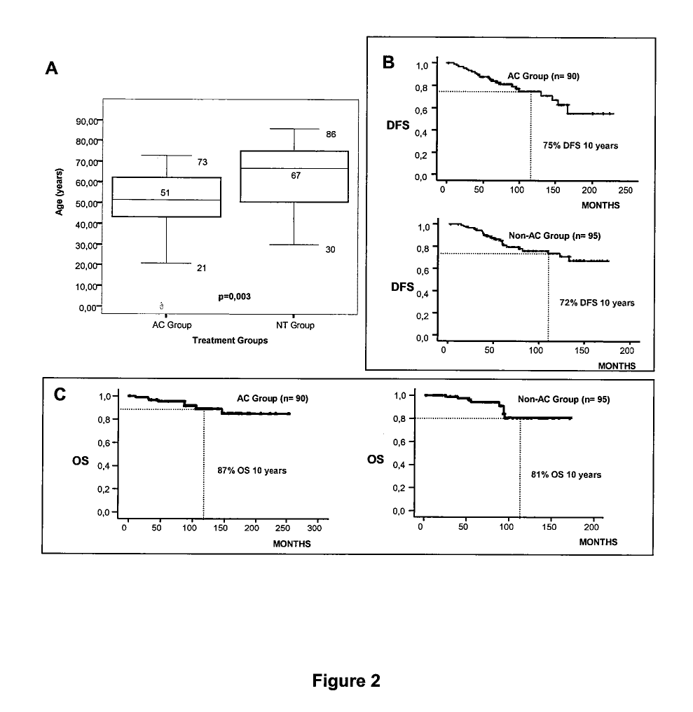

To define the genomic profile of NNBC, a genome-wide array CGH analysis of 185 tumors was performed. Clinico-pathological characteristics of the patients and tumors are summarized in Table 2 and FIG. 2. They were selected among a cohort of over 400 NNBC patients presenting to a single institution from which follow-up time was longer than 4 years. Based on the clinico-pathological features of the patients, 90 women received anthracycline-based chemotherapy (AC group) whereas 95 patients did not (non-AC group). In both groups, women with ER/PR positive tumors were treated with tamoxifen: 42 in the AC group (47%) and 56 in the non-AC group (59%). With a median duration of follow-up time of 82 months (range, 9 to 218 months), 45 of the 185 patients (24%) have relapsed. Median duration of follow-up time for patients who are free of disease was 96 months in both AC and non-AC groups. Death from the disease was assessed in 16 of 185 patients (9%).

Differences in clinico-pathological features and outcome between patients in the AC group vs. those in the non-AC group were determined. Women in AC group were younger (mean, 51 vs. 67 years; p=0.003) and had a more frequent pre-menopausal status (43 vs. 17%; p=0.001) (FIG. 2A). Statistically significant differences in any other histopathological feature were not observed. However, tumors in the non-AC group were slightly smaller, were more commonly classified as histological grade I, and expressed more frequently ER and PR (Table 2). Of the 45 patients who relapsed, 23 (26%) were included into the AC group (median duration of follow-up, 85 months; range, 9 to 218) whereas 22 (23%) were included into the non-AC group (median duration of follow-up, 77 months; range, 11 to 174 months). Of the 16 patients who succumbed to the disease, 8 were in the AC group and 8 were in the non-AC group. No statistically significant differences in DFS and OS were found between patients in AC group vs. non-AC group (FIGS. 2B and 2C).

2.2 Genomic Profiling of Lymph-Node Negative Breast Cancer

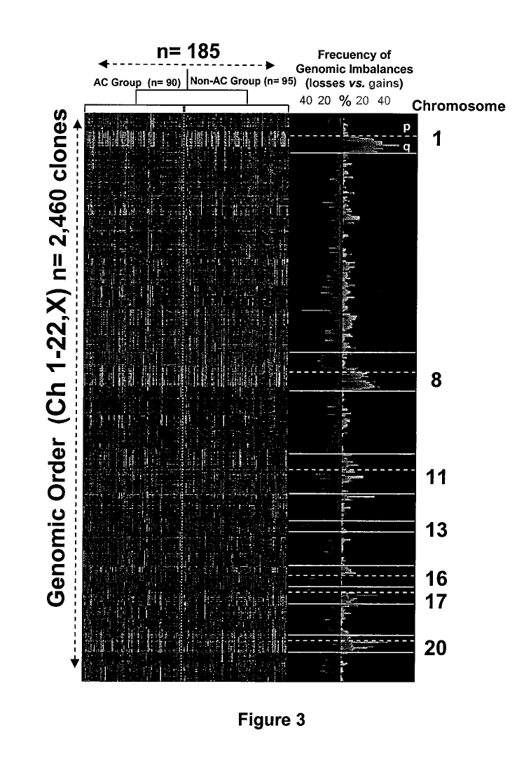

Array CGH analysis was successfully performed in the 185 tumor biopsies, all of which showed genomic alterations. The median number of abnormal clones per tumor was 188 (range, 1-1,280), which represent 8% of the total number of clones that were efficiently hybridized (range, 0.1-56%). These included 96 overrepresented clones (range, 1-178) and 92 deleted clones (range, 1-527) (FIG. 3).

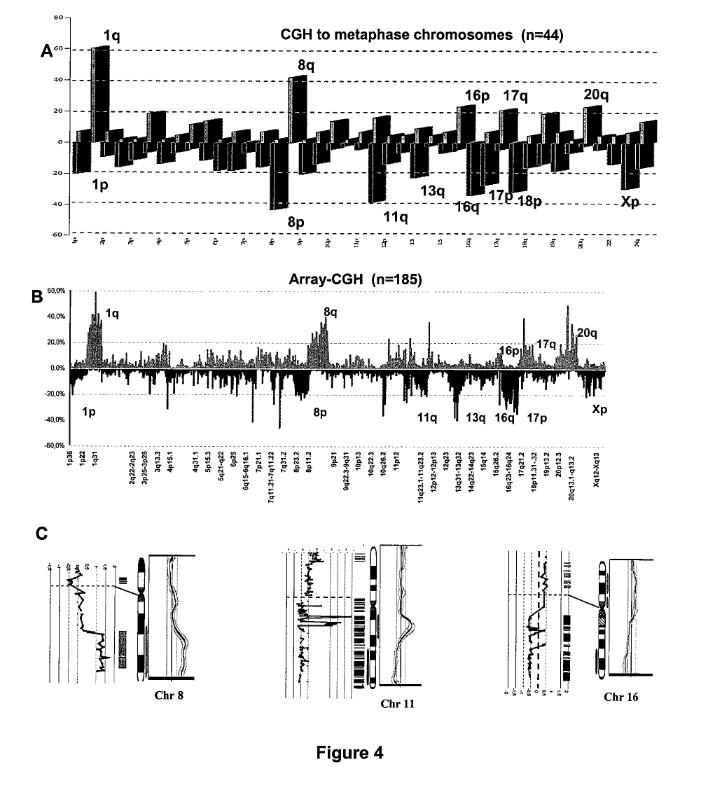

To initially validate the array CGH results, a subset of 44 samples was also analyzed with CGH to chromosomes, and the two techniques showed concordant values (see FIG. 4). To further validate these data, the analysis of 9 individual BAC clones in 22 frozen tumor sections using FISH also showed a high concordance with array CGH results (Table 3).

TABLE 3
FISH analysis of frozen tumor samples. Correlation with array CGH results

| | | | | | | Array_CGH | |
|---|---|---|---|---|---|---|---|
| Id_sample | | | | Cyto- | | log2rat | Genomic |
| (1) | Group | Clon_probe | Kb_position (2) | genetic_band | Gene | (3) | change (4) |
| 7059RGS | Training | RP11-113B7 | chr8: 4,218,951-4,386,750 | 8p23.2 | | −0.517101 | D |
| 7059RGS | Training | centromere | chr8: 46,070,002-47,036,668 | 8cen | | x | X |
| 7059RGS | Training | CTD-2013D21 | chr8: 110,489,880-110,668,770 | 8q23.1 | EBAG | 0.568455 | G |
| 1880EGF | Validation | RP11-113B7 | chr8: 4,218,951-4,386,750 | 8p23.2 | | −0.644832 | D |
| 1880EGF | Validation | centromere | chr8: 46,070,002-47,036,668 | 8cen | | x | X |
| 1880EGF | Validation | CTD-2013D21 | chr8: 110,489,880-110,668,770 | 8q23.1 | EBAG | 0.863346 | G |
| 3241RMB | Training | CTD-2192B11 | chr11: 69,070,148-69,070,609 | 11q13 | CCND1 | 0.032222 | N |
| 3241RMB | Training | CTD-2059P15C | chr11: 112,785,547-112,800,616 | 11q23 | DRD2 | −0.634542 | D |
| 333QOR | Training | CTD-2192B11 | chr11: 69,070,148-69,070,609 | 11q13 | CCND1 | −0.449662 | D |
| 333QOR | Training | CTD-2059P15C | chr11: 112,785,547-112,800,616 | 11q23 | DRD2 | −0.420051 | D |
| 333QOR | Training | RP11-17M17 | chr11: 132,052,507-132,205,819 | 11q25 | OPCML | −0.445597 | D |
| 333QOR | Training | centromere | chr11: 53,565,002-54,831,668 | 11qcen | | x | X |
| 7099NBL | Training | RP11-224E17 | chr16: 67,376,000-67,426,927 | 16q22 | CDH1 | −0.44705 | D |
| 7099NBL | Training | centromere | chr16: 35,666,668-36,933,334 | 16cen | | x | X |
| 5849MCP | Training | RP11-224E17 | chr16: 67,376,000-67,426,927 | 16q22 | CDH1 | −0.353041 | D |
| 5849MCP | Training | centromere | chr16: 35,666,668-36,933,334 | 16cen | | x | X |
| 7222CTD | Training | RP11-199F11 | chr17: 7482208-7462284 | 17p13.1 | TP53 | −0.643703 | D |
| 7222CTD | Training | RP11-400F19 | chr17: 37,857,983-36,058,331 | 17q21.2 | WI-14373 | x | X |
| 7222CTD | Training | centromere | chr17: 22,200,001-22,800,000 | 17cen | | x | X |

| | FISH | | | |
|---|---|---|---|---|
| | Genomic | | | |
| Id_sample (1) | change (4) | Copy_number | Frequency | Cells evaluated |
| 7059RGS | D | 1 | 47% | 38 |
| 7059RGS | G | 5 | 47% | 38 |
| 7059RGS | x | | | |
| 1880EGF | N | 2 | 30% | 50 |
| 1880EGF | G | 3 | 30% | 50 |
| 1880EGF | x | x | x | x |
| 3241RMB | N | 2 | 84% | 100 |
| 3241RMB | D | 1 | 100% | 100 |
| 333QOR | D | 1 | 80% | 100 |
| 333QOR | D | 1 | 80% | 100 |
| 333QOR | D | 1 | 70% | 100 |
| 333QOR | D | 1 | 70% | 100 |
| 7099NBL | D | 1 | 10% | 100 |
| 7099NBL | N | 2 | 10% | 100 |
| 5849MCP | D | 1 | 30% | 100 |
| 5849MCP | N | 2 | 30% | 100 |
| 7222CTD | D | 1 | 100% | 100 |
| 7222CTD | N, G | 2, 3 | 80%, 15% | 100 |
| 7222CTD | N | 2 | 100% | 100 |

(1) The probes have been tested in normal breast tissues getting as result 2 copies number each one in a minimum of 50 cells.
(2) Based on UCSC Genome Browser on Human May 2004 Assembly Version at http://genome.ucsc.edu
(3) Log2rat between −0.3 and 0.3 used to be normal values that correspond to no copy number changes. Log2rat lower than −0.3 correspond to deletions. Log2rat higher than 0.3 correspond to DNA copy number gains.
(4) N = normal, no copy number changes. D = deletion. G = Gain.

To define the common genomic signature of NNBC, clones that showed abnormal log2 ratios in more than 15% of the samples were searched for. A total of 112 clones that were mapped to 40 different chromosome loci in 9 different chromosome arms were found (FIG. 3). These corresponded to 23 genomic gains and 17 genomic losses involving regions known to be commonly involved in breast cancer as well as uncharacterized genomic aberrations. The most common gains corresponded to chromosomes 1q31 and 20812 (91 of 185, 49%), 8824.2 (40%), 17g21 (39%), 1q32, 8823.1 and 20813.1 (35%), 1 q23 (34%) and 8824.1 at MYC gene locus (32%). In addition, high-level amplification (defined as log2 ratio>1 observed in at least 10 different samples) was identified in 5 different regions of chromosomes 11g13-q14 at CCND1 gene (17 of 185 tumors, 9%), HER2 (13 tumors, 7%), 1g31 and 8p12 at FGFR1 frequently deleted regions were observed at chromosomes 13814-q22 (66 of 185 tumors, 36%), 17p12-p13 including P53 gene locus (34%), 16821-q22 including the CDH1 gene (30%), and 11g21-q25 (29%), 16824 and 16p12-p13.1 (26%), 11g12 (25%), 8p21.3-p22 (25%) and 22g11.2. A total of 18 homozygous deletions (defined as log2 ratio below −1.4) were identified, being the loss of 13g21.3-q22 at KLF12 gene observed in two different tumors. A detailed delineation of the regions of DNA copy number change with genes targeted by genomic aberrations is shown in Table 4.

TABLE 4
Description of common regions of genomic gain and amplification, hemizygous loss
and homozygous deletion in lymph node negative breast tumors. Mb position is based
on UCSC Genome Browser Human May 2004 version, http://genome_{:}cse.ucsc.edu
A.- Frequent genomic losses

| Cytogenetic_band | Clone | Mb Positión | Sample (N) | Candidate Gene |
|---|---|---|---|---|
| 1p36 | RP11-265F14 | 15.5 | 33 | CASP9 |
| 4p12 | RP11-38M16 | 40.1 | 32 | CHRNA9 |
| 8p12 | RP11-277I21 - RP11-57I3 | 28.9-32.5 | 36 | KIF13B o GAKIN, NRG1 |
| 8p21.3-p22 | RP11-107P5 | 17.4 | 44 | PDGFRL, MTSG1 |
| 8p22-8p23 | RP11-235O5 | 10.5 | 35 | SOX7 |
| 8p23.3 | RP11-82K8 | 2.1 | 36 | |
| 11q12 | RP11-77M17 - RP11-548G17 | 57.1-64.5 | 45-47 | |
| 11q22.3-11q24 | RP11-759M17 - RP11-87O12 | 110.9-122.7 | 34-38 | PPP2R1B, DRD2 |
| 11q25 | RP11-17M17 | 132 | 33 | OPMLC |
| 13q14.2 | RP11-120G8 | 47.4 | 66 | MED4 VDRIP |
| 13q21.3-13q22 | RP11-31C6 - RP11-9P22 | 72.6-73.6 | 45-60 | KLF12 |
| 16p12-16p13.1 | RP11-109D4 | 18.6 | 48 | SMG1 |
| 16q21-q22 | RP11-5A19 - RP11-123C5 | 65.6-67.5 | 40-56 | TRADD, CTCF, CDH1 |
| 16q24 | RP11-140K16 - RP11-59A12 | 82.9-83.4 | 48 | |
| 17p12 | CTB-194B18 - RPC-34H11 | 9.7-15.9 | 48-64 | NCOR1 |
| 17p13 | RP1-172N16 - RP1-89K1 | 2.4-7.4 | 45-61 | TP53 |
| 22q11.2 | RP11-22M5 | 20.6 | 39 | PPM1F |

B.- Homozygous deletions

| | | | | | Tamaño |
|---|---|---|---|---|---|
| Cytogenetic_band | Clone | Mb Positión | Sample (N) | Candidate Gene | (Kb) |
| 2p21 | RP11-130P22 | chr2: 46,354,971-46,464,401 | 1 | EPAS-1 | 109.431 |
| 2p22.3 | RP11-444D15 | chr2: 32,074,776-32,230,735 | 1 | | 155.96 |
| 4p16 | RP11-97H19 | chr4: 6,879,991-6,900,849 | 1 | | 20.859 |
| 5 p tel | RP1-24H17 | chr5: 1-634,440 | 1 | AHH, AHHR | 634.44 |
| 6p21.2 | RP11-14G23 | chr6: 40,452,868-40,632,593 | 1 | LRFN2 | 179.726 |
| 7 p tel | RP1-164D18 | chr7: 1-836,351 | 1 | | 836.351 |
| 7q31.1 | RP11-77E2 | chr7: 107,112,166-107,279,614 | 1 | | 167.449 |
| 8p23.2 | RP11-113B7 | chr8: 4,218,951-4,386,750 | 1 | CSMD1 | 167.8 |
| 9p23 | RP11-50C21 | chr9: 10,400,067-10,554,235 | 1 | | 154.169 |
| 11q13.4 | CTD-2080I19 | chr11: 68,427,947-68,464,635 | 1 | IGHMBP2 | 36.689 |
| 11q24 | RP11-20M1 | chr11: 125,827,487-125,907,004 | 1 | KIRREL3 | 79.518 |
| 11 q tel | RP1-26N8 | chr11: 133,800,001-134,452,384 | 1 | | 652.384 |
| 13q21-13q22 | RP11-31C6 | chr13: 72,628,311-72,628,651 | 1 | | 0.341 |
| 13q21.3-13q22 | RP11-46L3 | chr13: 73,510,380-73,672,828 | 2 | KFL12 | 162.449 |
| 13q21-13q22 | RP11-9P22 | chr13: 73,620,422-73,769,921 | 1 | KFL12 | 149.5 |
| 13 q tel | RP1-01L16 | chr13: 113,800,001-114,142,980 | 1 | CDC 16 | 342.98 |
| 19q13.2 | RP11-18J23 | chr19: 48,075,707-48,076,061 | 1 | PSG1, PSG3 | 0.355 |

| | | | | |
|---|---|---|---|---|
| Cytogenetic_band | Clone | Mb Positión | Sample (N) | Candidate Gene |

C.- Frequent genomic gains

| | | | | |
|---|---|---|---|---|
| 1q23 | RP11-4J2 | 174 | 64 | |
| 1q31 | RP11-243M13 | 201.6 | 91 | RBBP5 |
| 1q32 | RP11-66M7 | 213.5 | 65 | ESRRG |
| 3q27-3q28 | CTD-2091K6 | 188.8 | 33 | BCL6 |
| 8q23.1 | CTD-2013D21 | 110.5 | 65 | EBAG9 |
| 8q24.12-8q24.2 | RP11-229L23 | 125.6 | 55 | MMTS1 |
| 8q24.1 | RP11-145G10 - | 128.6-128.7 | 60 | MYC |
| | DMPC-HFF#1-71E5 | | | |
| 8q24.2 | RP11-128P9 | 133.6 | 74 | KCNQ3 |
| 8q24.2 | RP11-184M21 | 134.1 | 62 | TG, SLA1 |
| 11p15.3-11p15.4 | RP11-28I11 | 10.5 | 43 | |
| 11q13 | RP1-88B16 | 69.1 | 44 | CCND1 |
| 11q13 | RP1-4E16 | 69.1 | 44 | CCND1 |
| 16p11.2 | RP11-146J7 | 25.7 | 32 | |
| 17q12 | DMPC-HFF#1-61H8 | 35.1 | 29 | ERBB2 |
| 17q21 | CTB-305D20 | 42.1 | 73 | WNT3, WNT9B |
| 17q22 | RP11-143M4 | 47.6 | 34 | |
| 17q22 | RP11-131C4 | 47.6 | 30 | |
| 17q24 | RP11-84E24 | 67.5 | 33 | SOX9 |
| 17q24 | RP11-128J1 | 74.8 | 35 | |
| 20p12 | CTD-2013D15 | 10.6 | 35 | JAG1 |
| 20q12 | RP11-93L19 | 39 | 71 | |
| 20q12 | RP11-13F12 | 40.4 | 91 | PTPRT |
| 20q13.1 | RP11-51K19 | 46.7 | 65 | PREX1 |

D.- Genomic amplifications

| | | | | |
|---|---|---|---|---|
| 1q31 | RP11-243M13 | 20.1 | 11 | RBBP5 |
| 8p12 | RP11-100B16 - | 38.3 | 9-10 | FGFR1 |
| | RP11-265K5 | | | TD52, STK3, |
| 8q21-8q24.2 | RP11-107F3 - | 74.3-138.7 | 7-10 | MST2, EBAG9, EXT1, |
| | RP11-122H7 (21 clones) | | | MTSS1, MYC, TG, SLA1 |
| 11q13 | RP1-88B16 | 69.1 | 17 | CCND1 |
| 11q13 | CTB-36F16 | 69.3 | 11 | FGF3 |
| 11q13 | CTC-437H15 | 69.9 | 8 | EMS1, CTTN |
| 11q13-14 | GS-7N12 | 76.7 | 13 | PAK1 |
| 17q12 | DMPC-HFF#1-61H8 | 35.1 | 13 | ERBB2 |

2.3 Correlation of Genomic Alterations and Clinicopathological Features

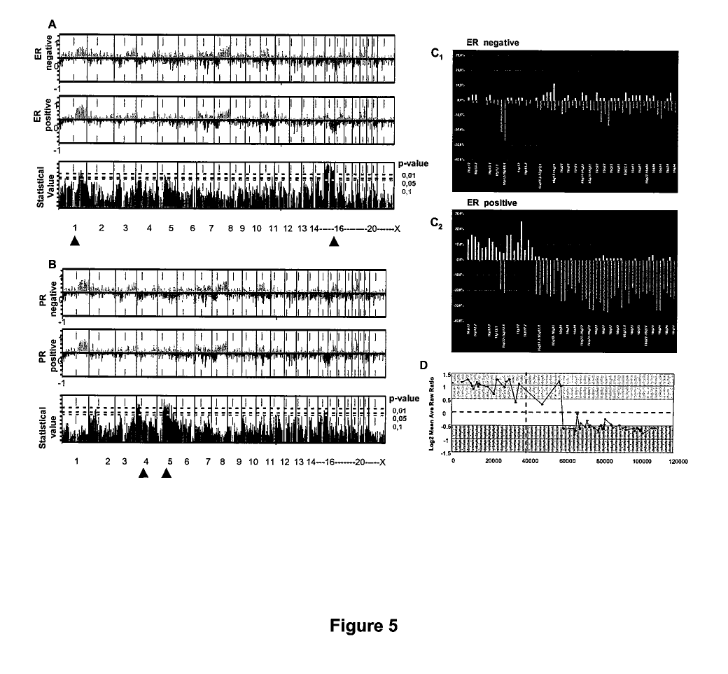

The association of clinical and pathological variables (age, clinical stage, tumor size, histological grade and subtype, hormonal status and ER and PR status) with each of the BAC clones in the 185 patients was tested. After adjustment for multiple testing, these analyses showed that the only variables correlated with genomic changes were ER and PR status (FIG. 5). Tumors that showed expression of ER (ER+) presented with frequent gain of chromosomes 1q21-q43 (35% vs. 14%; p<0.05), and 16p12 (17% vs. 1%; p<0.01) and losses of chromosome 16q21-q24 (25% vs. 7%; p<0.01). Tumors negative for PR (PR−) also presented with frequent deletion of chromosomes 4p13-p16 (19 vs. 5%; p<0.001) and 5q11.2-q31 (16 vs. 3%; p<0.001) (FIG. 5B). Because HER2 gene amplification (and/or over-expression) has been accepted at the most recent St Gallen criteria as a risk prognostic factor in NNBC, the correlation of the genomic status of 17q12 locus at HER2 gene (determined by array CGH analysis) with other clinical variables was determined. Amplification or gain of HER2 gene was observed in 29 tumors (16%) and was correlated with negativity for PR expression (p=0.007), but not with other clinico-pathological features.

2.4 Association of Genomic Abnormalities with Clinical Outcome: 11q Loss Predicts Response to Chemotherapy

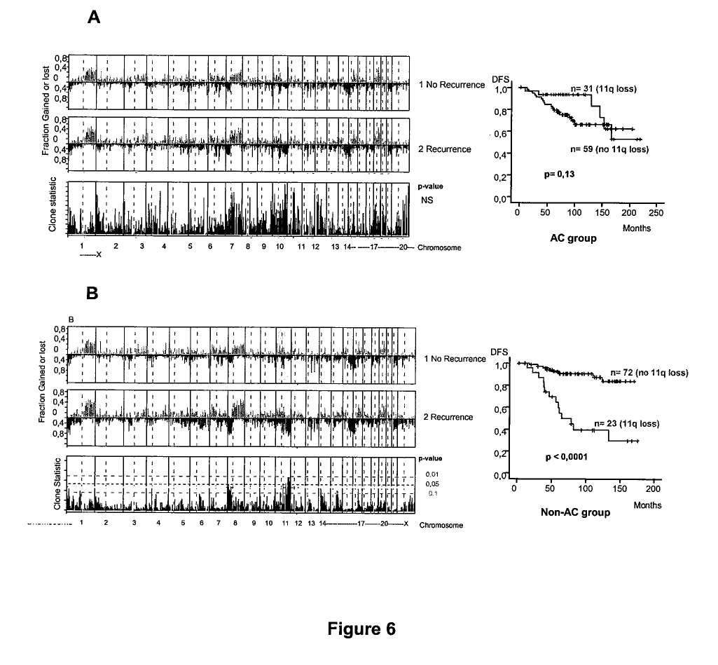

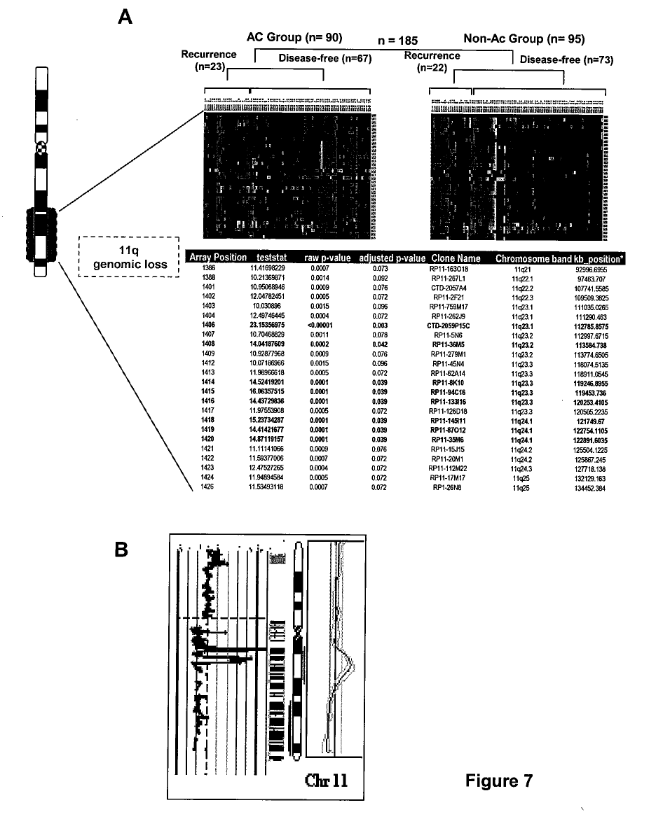

Because systemic chemotherapy results in improvement of DFS and OS in patients with breast cancer, the authors hypothesized that the genomic tumor profile conditioned response to chemotherapy in the series. To test this hypothesis, patients were separated into two treatment cohorts based on whether they received treatment with anthracycline-based chemotherapy (AC group) or not (non-AC group). In the series, none of the classical prognostic factors in NNBC (age, clinical stage, tumor size, histological grade and subtype, hormonal status and ER and PR status) were correlated with disease-free survival (DFS) in both AC and non-AC groups (FIG. 2). In addition, a similar relapse rate was observed in the differently treated subgroups: 23 of 90 patients (26%) had tumor recurrence in the AC group vs. 22 of the 95 patients (23%) in the non-AC group. The genomic profiles of tumors in the AC and non-AC groups were then compared. None of the abnormal BAC clones showed a significantly different distribution between the two cohorts, indicating that both groups were comparable at the genomic level (FIG. 3). To develop a genomic predictor of clinical outcome in patients with NNBC, the association of the genomic aberrations with recurrence of the disease in the two differently treated cohorts was examined. In the AC group, after adjustment for multiple testing, none of the abnormal BAC clones was associated with tumor relapse (FIG. 6A). In the non-AC group, however, there were statistically significant differences (p<0.05) in 8 BAC clones that showed more common deletion in tumor recurrences with respect to non-recurrences (FIG. 6B). Notably, these 8 clones clustered to the long arm of chromosome 11 from 11q23.1 to 11q24.1, spanning ˜9 Mb. in size. If it is considered the adjusted value for statistical significance of <0.1 instead of 4.05, the number of BAC clones correlated with relapse increases to 24, all of them mapped to 11q21-q25 and covering a larger region of ˜35 Mb. in size (FIG. 7).

Therefore, deletion of chromosome 11q was associated with decreased DFS in NNBC patients in the non-AC group (DFS±SE at 10 years, 40±14% vs. 86±6%, p<0.0001). On the contrary, in the non-AC group, patients with 11q loss presented a lower relapse rate than those without 11q deletion, although this difference did not reach a statistically significant value (DFS at 10 years±SE, 92±21% vs. 65±9%, p=0.13). Considering the patients harbouring deletion of chromosome 11q, five of 31 patients in the AC group (16%) had recurrence of the disease whereas the relapse rate was much higher in the non-AC group: 14 of 23 with 11q deletion (62%) had a relapse (p<0.0001). Among the 59 patients in the AC group who did not show deletion of 11% 19 (30%) presented recurrence of the disease whereas only 8 of 72 without 11q deletion (11%) in the non-AC group relapsed. Analysis of the association of the genomic changes with OS in the two treatment cohorts did not reveal any significant correlation, probably due to the low number of patients who have died of the disease so far.

Finally, a statistical analysis to check if there were any GO categories that were enriched in the genes located in the region of deletion in chromosome 11q23.1-q24.1 was performed. Among them, DNA repair genes and meiotic-related genes were significantly enriched (hypergeometric test p value<0.00092). Four genes belonged to this category (CHK1, H2A, ATM and ZW10).

2.5 Characteristics of Patients with 11q Deletions

To determine whether the negative impact of 11q deletion on DFS was dependent on other clinical and biological features, the clinical and biological characteristics of the 54 patients with 11q deletion vs. those 131 patients without deletion of 11q was compared. In the whole group of 185 patients, there were no statistically significant differences for age, clinical stage, hormonal status, tumor size and grade, and expression of ER/PR for 11q-deleted vs. non-deleted tumors. Similarly, there were no differences in these variables when the patients were separated in the AC and non-AC groups (Table 5). In CCND1 and HER2 amplification subgroups, no separate statistical analysis for AC and non-AC groups could be performed because of the small number of patients. These data indicate that the influence of 11q deletions in the relapse rate of the patients in the non-AC group is independent of other known clinical and pathological features. Possible differences in patients with and without 11q deletion for specific genetic alterations which are correlated with clinically aggressive breast cancer (HER2, CCND1, MYC and FGFR1 amplifications and P53 and P16 deletions) was also analyzed. Changes in the distribution of these genomic alterations were not observed, with the exception of CCND1 amplification that was more common in tumors harboring deletion of chromosome 11q: among 17 cases with CCND1 amplification, 12 (70%) presented deletion of 11q whereas only 42 cases (25%) showed 11q deletion among the 168 non-amplified CCND1 cases (p<0.001). This association can probably be explained by the proximity of CCND1 gene (which maps to 11q13 band) to the 11q23.1-q24.1 deletion, as it is widely accepted that unrepaired or misrepaired DNA double strand breaks lead to the formation of contiguous chromosome amplifications, deletions and translocations in human cancer (FIG. 7B). Notably, genomic amplification of CCND1 was not associated with decreased DFS in both the non-AC and AC groups. In summary, tumors with 11q deletion do not show a more aggressive phenotype or genotype that can distinguish them from those without this chromosome deletion.

TABLE 5
Clinico-pathological and genetic characteristics, and survival rates of patients with 11 q deletion vs. those without 11 q deletion

| | | ALL PATIENTS (n = 185) | | | | AC GROUP (n = 90) | | | | NON-AC GROUP (n = 95) | | | |
|---|---|---|---|---|---|---|---|---|---|---|---|---|---|
| | | LOSS OF 11 q | | | | LOSS OF 11 q | | | | LOSS OF 11 q | | | |
| | | NO | YES | n | p-value | NO | YES | n | p-value | NO | YES | n | p-value |
| Age (years) | <35 | 64% | 36% | 11 | NS | 56% | 44% | 9 | NS | 100% | 0% | 2 | NS |
| | 35-50 | 68% | 32% | 54 | | 65% | 35% | 32 | | 73% | 27% | 22 | |
| | 51-55 | 81% | 19% | 16 | | 77% | 23% | 13 | | 100% | 0% | 3 | |
| | >55 | 71% | 29% | 104 | | 64% | 36% | 36 | | 75% | 25% | 68 | |
| Hormonal Status | Postmenopausic | 71% | 28% | 55 | NS | 66% | 32% | 48 | NS | 76% | 24% | 67 | NS |
| | Premenopausic | 72% | 29% | 115 | | 68% | 34% | 39 | | 80% | 20% | 16 | |
| Tumoral Size | T1 (<2 cm) | 73% | 27% | 70 | NS | 67% | 33% | 27 | NS | 77% | 23% | 43 | NS |
| | T2 (2-5 cm) | 69% | 31% | 10 | | 68% | 32% | 56 | | 70% | 30% | 44 | |
| | T3 (>5 cm) | 60% | 40% | 100 | | 33% | 64% | 6 | | 100% | 0% | 4 | |
| Stage | I | 73% | 27% | 68 | NS | 63% | 37% | 24 | NS | 79% | 21% | 44 | NS |
| | II | 68% | 32% | 110 | | 67% | 33% | 64 | | 70% | 30% | 46 | |
| Hormonal Receptors | ER+ | 68% | 32% | 101 | NS | 62% | 38% | 42 | NS | 73% | 27% | 59 | NS |
| | ER− | 78% | 22% | 60 | | 73% | 27% | 33 | | 85% | 15% | 27 | |
| | PgR+ | 71% | 29% | 96 | NS | 60% | 40% | 43 | NS | 79% | 21% | 59 | NS |
| | PgR− | 74% | 26% | 65 | | 75% | 25% | 32 | | 70% | 30% | 33 | |
| Histologic Grade | I | 77% | 23% | 41 | NS | 75% | 25% | 13 | NS | 78% | 22% | 28 | NS |
| | II | 66% | 34% | 90 | | 59% | 41% | 44 | | 72% | 28% | 46 | |
| | III | 80% | 20% | 17 | | 71% | 29% | 12 | | 100% | 0% | 5 | |
| Recurrence | no | 75% | 25% | 140 | 0.037 | 61% | 39% | 67 | NS | 88% | 12% | 73 | <0.0001 |
| | yes | 58% | 42% | 45 | | 78% | 22% | 23 | | 36% | 64% | 22 | |
| CCND1 amplification | no | 75% | 25% | 167 | <0.001 | 70% | 30% | 80 | 0.008 | 79% | 21% | 87 | 0.02 |
| | yes | 30% | 70% | 17 | | 22% | 78% | 9 | | 37% | 63% | 8 | |
| HER2neu amplification | no | 71% | 29% | 164 | NS | xxx | xxx | xxx | * | xxx | xxx | xxx | * |
| | yes | 69% | 31% | 13 | | xxx | xxx | xxx | | xxx | xxx | xxx | |
| C-MYC amplification | no | 72% | 28% | 175 | NS | xxx | xxx | xxx | * | xxx | xxx | xxx | * |
| | yes | 44% | 56% | 9 | | xxx | xxx | xxx | | xxx | xxx | xxx | |
| P53 deletion | no | 76% | 24% | 103 | NS | 70% | 30% | 52 | NS | 80% | 20% | 55 | NS |
| | yes | 64% | 36% | 45 | | 71% | 29% | 14 | | 61% | 39% | 31 | |

2.6 Validation of 11q Deletion as a Therapy-Predictive Indicator of Clinical Outcome in Early Breast Cancer

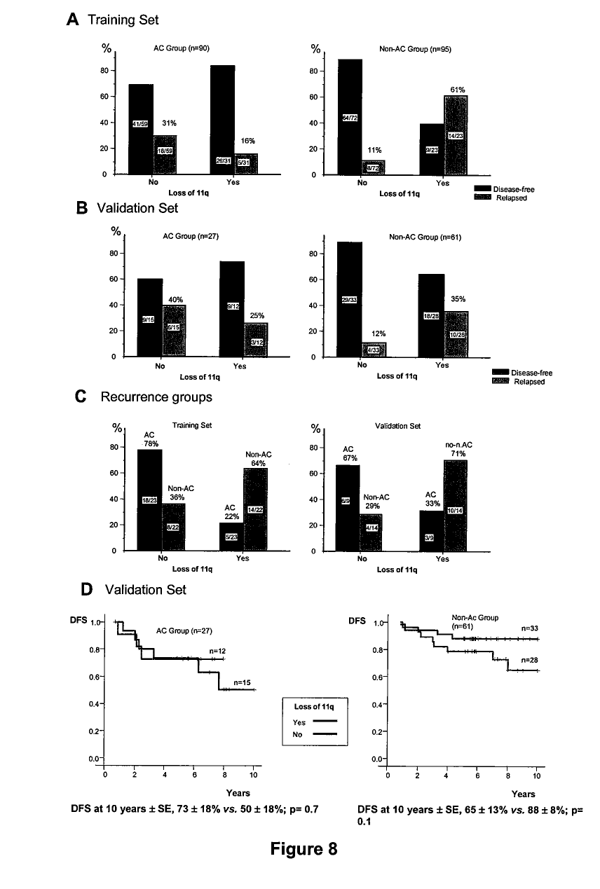

To validate the association of chromosome 11q deletion with worse outcome in patients not receiving anthracycline-based chemotherapy, a second series (validation group) of 88 tumor biopsies from an independent cohort of NNBC patients was analyzed. These included 18 Spanish and 70 American patients who had comparable clinico-pathological features and treatments to the initial group of 185 patients (Table 2). After surgery, 27 of the patients received chemotherapy whereas the remaining did not. Sixty-two patients with ER/PR positive expression received hormonal therapy based on tamoxifen. Determination of 11q status was performed using BAC array CGH. In the group treated with chemotherapy, 6 of 15 patients (40%) without 11q deletion relapsed whereas 3 of 12 patients (25%) with 11q deletion had a recurrence (p=0.23). However, in the group not receiving chemotherapy, tumor recurrence was observed in 4 of 33 patients (12%) without 11q deletion and in 10 of 28 patients (35%) with lq deletion (p=0.02) (FIG. 8A-C). Kaplan-Meier curves also showed that deletion of chromosome 11q was associated with inferior DFS in patients not treated with chemotherapy (DFS±SE at 10 years, 65±13% vs. 88±8%, p<0.1). Notably, in the non-AC group, patients with 11q deletion had a tendency to show a superior DFS compared to those without 11q loss (73±18% vs. 50±18%, p<0.7). These differences, however, did not reach statistically significant values, probably because of the limited number of patients and the relatively short median follow-up time (FIG. 8D). In summary, the results observed in this validation set were coincident with the results obtained in the training set of 185 patients, and confirms that deletion of 11q is associated with relapse in patients with NNBC who are not treated with anthracycline-based chemotherapy

Discussion

Following current therapeutic guidelines, one fourth of NNBC patients will have tumor recurrence and ultimately die of the disease. In addition, many patients treated with systemic therapy who will never have disease recurrence could have been cured with surgery alone. These over- and under-treatments are owing to limitations of the current prognostic factors, which largely rely on clinical characteristics and classical histopathological features. Recently, HER2 amplification/over-expression has been accepted as a risk factor for prognostication in the St Gallen criteria and moreover, recent reports demonstrate that a recombinant monoclonal antibody against HER2 combined with chemotherapy improves outcomes among women with HER2-positive breast cancer. Still, as this therapy will benefit ˜20% of NNBC women with HER2-positive tumors, there is an urgent need of similar therapy-predictive factors to tailor optimal individualized therapies in the remaining women. In the present invention, by using CGH to BAC microarrays for scanning NNBC genomes, the single deletion of chromosome 11q has been identified as a novel genomic marker that predicts response to anthracycline-based chemotherapy. Thus, patients receiving anthracycline-based chemotherapy with 11q deletion had lower tumor relapse rates than those not having 11q deletion. On the contrary, in the group of patients not receiving chemotherapy, tumors with 11q deletion relapsed more frequently than those without 11q loss. Notably, the presence of 11q deletion in tumors was not correlated with classical prognostic factors such as age, clinical stage, tumor size, histological grade and subtype, and ER and PR expression status, or with other genetic alterations correlated with poor outcome in breast cancer (HER2, MYC and FGFR1 amplification and P53 and P16 deletion). Thus, the adverse outcome of 11q deletion in the non-AC group was independent of all tested prognostic factors. Therefore, the data herewith presented suggest that NNBC patients with 11q deleted tumors may benefit from the use of systemic chemotherapy that could be considered as the first treatment option for these patients despite other clinical, histopathological and genetic characteristics.

In the attempt of delineating the minimal region of common loss of 11q, it has been observed that most tumors in this study showed large 11q deletions extending from bands 11q21 to 11q25.

The bottom line of the findings of the inventors is that the loss of chromosome 11q makes tumor cells responsive to anthracycline-based chemotherapy. Why 11q-deleted cells become more sensitive to chemotherapy is currently unknown.

Finally, the invention could be valuable in the clinical management of patients with NNBC, by adding the 11q deletion status to the currently accepted prognostic and therapy-predictive markers. Accordingly, tumors should be screened for the presence or absence of 11q deletion at diagnosis using appropriate techniques such as rapid quantitative PCR, FISH and/or mini-array CGH devices using a reduced set of BAC clones. These diagnostic tests should allow clinicians to prospectively identify patients who are candidates to receive anthracycline-based chemotherapy, such as standard AC/FAC, which are widely used as frontline therapies in NNBC, irrespective of other clinico-pathological features. In patients presenting factors that imply a good prognosis, such as age>35, clinical stage I, low-grade tumors sized>1 cm, and ER/PR positivity, systemic chemotherapy could be avoided only if 11q deletion is not identified.

1. An in vitro method for predicting sensitivity and response to chemotherapy in a patient with a breast cancer, said method comprising:
providing a breast tumor sample from said patient;
obtaining a nucleic acid present in said sample; and
detecting the presence or absence of a deletion in the human chromosome region 11q21-q25;
wherein the presence of said deletion is indicative of a favourable predisposition of said patient to respond to a chemotherapy treatment, and wherein the presence of said deletion may be used to design an individual chemotherapy for said patient, and/or to minimize the relapse risk by administering chemotherapy to said patient, and/or to increase the survival rate of said patient by administering chemotherapy to said patient.

2. The method according to claim 1, wherein the presence of said deletion is indicative of a favourable predisposition of said patient to respond to an anthracycline-based chemotherapy treatment, and wherein the presence of said deletion may be used to design an individual anthracycline-based chemotherapy for said patient, and/or to minimize the relapse risk by administering an anthracycline-based chemotherapy to said patient, and/or to increase the survival rate of said patient by administering an anthracycline-based chemotherapy to said patient.

3. The method according to claim 1, wherein the deletion to be determined is located at the human chromosome region 11q23.1-q24.1.

4. The method according to claim 1, wherein the patient is a patient with lymph-node negative breast cancer or a patient with lymph-node positive breast cancer or a patient with metastatic breast cancer.

5. The method according to claim 1, wherein the detection of said deletion is carried out by a hybridization-based assay.

6. The method according to claim 5, wherein the detecting step comprises:
contacting the nucleic acid sample with one or more nucleic acid probes each of which selectively binds to a target polynucleotide sequence on the chromosome region 11q21-q25, under conditions in which the probe forms a stable hybridization complex with the target polynucleotide sequence; and
detecting the hybridization complex.

7. The method according to claim 6, wherein the step of detecting the hybridization complex comprises determining the copy number of the target polynucleotide sequence, thereby determining the presence of the deletion.

8. The method according to claim 5, wherein said hybridization assay is selected from the group consisting of Southern blot, LOH, PCR, in situ hybridization (ISH), fluorescence ISH (FISH) and comparative genomic hybridization (CGH).

9. The method according to claim 5, wherein the method is a comparative genomic hybridization assay.

10. The method according to claim 5, wherein said hybridization assay is an array-based assay.

11. The method according to claim 5, wherein said hybridization assay is an array-based CGH assay.

12. The method according to claim 1, which further comprises considering the data obtained for designing an individual chemotherapy treatment for said patient based on an anthracycline-based chemotherapy.

13. A kit for predicting sensitivity and response to chemotherapy in a patient with a breast cancer, said kit comprising one or more nucleic acid probes each of which selectively binds to a target polynucleotide sequence on the chromosome region 11q21-q25, under conditions in which the probe forms a stable hybridization complex with the target polynucleotide sequence.

14. The kit according to claim 12 wherein probe is directly labeled.

15. The kit according to claim 12 wherein said probe is indirectly labeled.

16. The kit according to claim 12 wherein the nucleic acid probe is attached to a solid surface.

17. The kit according to claim 15 wherein the attached probe is a member of a nucleic acid array.

18. The kit according to claim 12 wherein the kit further comprises instructional material which teaches that the detection of a deletion in the chromosome region 11q21-q25 in a cell from a breast tumor sample of a patient is indicative of a favourable predisposition of said patient to respond to a chemotherapy treatment.
